(19) United States
(12) Patent Application Publication
(10) Pub. No.: US 2016/0271284 A1
(43) Pub. Date: Sep. 22, 2016

(54) Ultraviolet High-Level Ultrasound Transducer Disinfection System

(71) Applicant: George J. Lichtblau, New Canaan, CT (US)

(72) Inventor: George J. Lichtblau, New Canaan, CT (US)

(21) Appl. No.: 15/169,943

(22) Filed: Jun. 1, 2016

Related U.S. Application Data
(63) Continuation of application No. 14/521,874, filed on Oct. 23, 2014.

(51) Int. Cl. A61L 2/26; A61L 2/10

(52) U.S. Cl. CPC A61L 2/26; A61L 2/10

(57) ABSTRACT

A UVC disinfection system which produces a dosage of UVC radiation sufficient to kill Clostridium difficile and other pathogens in a simple fast and unique manner. The system is especially useful for sterilizing and disinfecting medical ultrasound probes or transducers. The system in one embodiment comprises an enclosure for housing a plurality of UVC lamps which are disposed around a central area in which an ultrasound transducer can be disposed. The lamps are disposed in a vertical orientation, as is the transducer and provide 360° of UVC radiation around the transducer for effective and efficient decontamination. The UVC lamps can also be provided at the bottom of the enclosure. The system is controlled by an electronic controller which typically is a microprocessor based controller for control of the ballasts for the UVC lamps and having associated control switches and displays. The current input to the ballasts is monitored by the controller to assure that the UVC lamps are operating properly. A UVC radiation sensor provided in the housing monitors radiation intensity from the lamps. If the current and/or radiation intensity is below reference levels, the system is shut down by the controller.

### STATEMENT REGARDING FEDERALLY SPONSORED RESEARCH OR DEVELOPMENT

N/A

### BACKGROUND OF THE INVENTION

It has been well established that ultrasound transducers are a potential pathway to pass deadly pathogens between people. Therefore, it is important to reduce the probability of pathogens on the surface of the ultrasound transducer and its extended probe to a minimum.

External probes that only come into contact with clean, intact skin are considered noncritical devices and require cleaning after every use.

Internal probes should be covered with a single-use barrier, such as condoms. However, single-use disposable probe covers have been documented to have leakage between 0.9% and 81%. (Rutala and Weber, 2011).

For maximum safety, one should perform high-level disinfection of the probe between each use and use a probe cover or condom as an aid to keep the probe clean.

According to the Centers for Disease Control and Prevention (CDC) “Guideline for Disinfection and Sterilization in Healthcare Facilities” (2008):

“Cleaning is the removal of visible soil (eg, organic and inorganic material) from objects and surfaces and normally is accomplished manually or mechanically using water with detergents or enzymatic products. Thorough cleaning is essential before high-level disinfection and sterilization because inorganic and organic material that remains on the surfaces of instruments interfere with the effectiveness of these processes.”

“Disinfection describes a process that eliminates many or all pathogenic microorganisms, except bacterial spores.”

Low-Level Disinfection—Destruction of most bacteria, some viruses, and some fungi. Low-level disinfection will not necessarily inactivate Mycobacterium tuberculosis or bacterial spores.

Mid-Level Disinfection—Inactivation of M Tuberculosis, bacteria, most viruses, most fungi, and some bacterial spores.

High-Level Disinfection—Destruction/removal of all microorganisms except bacterial spores

“Sterilization describes a process that destroys or eliminates all forms of microbial life and is carried out in healthcare facilities by physical or chemical methods. Steam under pressure, dry heat, ethylene oxide (EtO) gas, hydrogen peroxide gas plasma, and liquid chemicals are the principal sterilizing agents used in health-care facilities. . . . When chemicals are used to destroy all forms of microbiologic life, they can be called chemical sterilants. These same germicides used for shorter exposure periods also can be part of the disinfection process (ie, high-level disinfection).”

The following specific recommendations are made for the cleaning and preparation of all ultrasound probes. Users should also review the CDC document on sterilization and disinfection of medical devices to be certain that their procedures conform to the CDC principles for disinfection of patient care equipment.

1. Cleaning—Transducers should be cleaned after each examination with soap and water or quaternary ammonium (a low-level disinfectant) sprays or wipes. The probes must be disconnected from the ultrasound scanner for anything more than wiping or spray cleaning. After removal of the probe cover (when applicable), use running water to remove any residual gel or debris from the probe. Use a damp gauze pad or other soft cloth and a small amount of mild nonabrasive liquid soap (household dish-washing liquid is ideal) to thoroughly cleanse the probe. Consider the use of a small brush, especially for crevices and areas of angulation, depending on the design of the particular probe. Rinse the probe thoroughly with running water, and then dry the probe with a soft cloth or paper towel.

2. Disinfection—As noted above, all internal probes (eg, vaginal, rectal, and transesophageal probes) as well as intraoperative probes require high-level disinfection before they can be used on another patient.

For the protection of the patient and the health care worker, all internal examinations should be performed with the operator properly gloved throughout the procedure. As the probe cover is removed, care should be taken not to contaminate the probe with secretions from the patient. At the completion of the procedure, hands should be thoroughly washed with soap and water. Gloves should be used to remove the probe cover and to clean the probe as described above.

Note: An obvious disruption in condom integrity does not require modification of this protocol. Because of the potential disruption of the barrier sheath, high-level disinfection with chemical agents is necessary. The following guidelines take into account possible probe contamination due to a disruption in the barrier sheath.

After removal of the probe cover, clean the transducer as described above. Cleaning with a detergent/water solution as described above is important as the first step in proper disinfection, since chemical disinfectants act more rapidly on clean and dry surfaces. Wet surfaces dilute the disinfectant.

High-level liquid disinfection is required to ensure further statistical reduction in the microbial load.

To achieve high-level disinfection, the practice must meet or exceed the listed “High-Level Disinfectant Contact Conditions” specified for each product. Users should be aware that not all approved disinfectants on this list are safe for all ultrasound probes.

The CDC recommends environmental infection control in the case of Clostridium difficile, consisting of “meticulous cleaning followed by disinfection using hypochlorite-based germicides as appropriate” (CDC, 2008). The current introduction and initial marketing of a hydrogen peroxide nanodroplet emulsion might provide an effective high-level disinfectant without toxicity.

The Occupational Safety and Health Administration as well as the Joint Commission (Environment of Care Standard IC 02.02.01 EP 9) have issued guidelines for exposure to chemical agents, which might be used for ultrasound probe cleaning. Before selecting a high-level disinfectant, users should request the Material Safety Data Sheet for the product and make sure that their facility is able to meet the necessary conditions to minimize exposure (via inhalation, ingestion, or contact through skin/eyes) to potentially dangerous substances. Proper ventilation, a positive-pressure local environment, and the use of personal protective devices (eg, gloves and face/eye protection) may be required.

Immersion of probes in fluids requires attention to the individual device's ability to be submerged. Although some scan heads as well as large portions of the cable may safely be immersed up to the connector to the ultrasound scanner, only the scan heads of others may be submerged. Some manufacturers also note that the crystals of the array may be damaged if, instead of suspending the probe in the disinfectant, it rests on the bottom of the container. Before selecting a method of disinfection, consult the instrument manufacturer regarding the compatibility of the to-be-used agent with the probes. Relevant information is available online and in device manuals. Additionally, not all probes can be cleaned with the same cleaning agents. Although some agents are compatible with all probes of a given manufacturer, others must be limited to a subset of probes.

After soaking the probe in an approved disinfectant for the specified time, the probe should be thoroughly rinsed (especially to remove traces of toxic disinfectants in the case of ortho-phthalaldehyde) and dried.

It has been well established that High-Level Disinfection is extremely important with the use of ultrasonic transducer probes. All of the present methods of disinfection, discussed above, rely upon chemicals which may be very dangerous to the health of a patient. An alternate technique is the use of high temperature steam; however, this may not be compatible with many of the ultrasound transducers.

An example of current technology to achieve high-level disinfection of an ultrasonic transducer is the Trophon EPR sold by General Electric and designed and produced by Nanosonics of Australia. This product uses vaporized hydrogen peroxide to decontaminate an ultrasonic transducer and takes about seven minutes for the decontamination cycle. Additional time is required to load the hydrogen peroxide container into the machine.

It is known that UVC radiation is effective in killing or deactivating pathogens in air, water and on exposed surfaces.

U.S. Pat. No. 8,791,441 of the same inventor as herein illustrates one method of producing ultraviolet radiation in the UVC band. In this case the product is designed to decontaminate the air and, at the same time, decontaminate the surrounding surfaces in a hospital environment, athletic locker room or the like.

The ability for UVC radiation to kill pathogens is determined by the dosage applied to a given area. Dosage is defined at the total energy that is applied to a given area and typically defined as Joules per square meter or Joules per square centimeter. The most common definition is “Watt-Second/Square Centimeter”. A “Watt-Second” is another name for a “Joule”. In dealing with infection control, a Watt-Second is an enormous amount of energy for a square centimeter; therefore, the common dosage is specified as either “Micro-Watt-Seconds/Square Centimeter”, or mJ/Square cm (1000 Micro-Watt-Seconds/Square CM). A Micro-Watt is one millionth of a watt.

As stated above, the CDC has stated that Clostridium difficile is particularly difficult to kill:

“The CDC recommends environmental infection control in the case of Clostridium difficile, consisting of “meticulous cleaning followed by disinfection using hypochlorite-based germicides as appropriate” (CDC, 2008). The current introduction and initial marketing of a hydrogen peroxide nanodroplet emulsion might provide an effective high-level disinfectant without toxicity.”

It is known that Clostridium difficile (C. Diff) is easier to kill using UVC Ultraviolet radiation, and it has been established that it takes between 30,000 and 60,000 micro-watt-seconds/square centimeter (30 to 60 mJ/square cm) to kill at least 99% of C. Diff.

There are many UVC products on the market that are designed to kill pathogens. For example:

US Patent application 2009/0304553 illustrates a method for sterilizing an item and storing the item in a sterile environment. It produces ozone, high intensity microwave radiation, or UVC to kill the pathogens.

Steril-Aire produces a SterilWand™ UVC Emitter™ which is used to decontaminate surfaces.

Nanoclave produces a Nanoclave Cabinet that uses UVC radiation to kill pathogens on hospital tools and equipment.

Air Science produced a “UV-Box” which uses UVC in an enclosed metal container to destroy exposed surface DAN and bacteria.

However, the above UVC equipment cannot guarantee decontamination of an ultrasonic transducer probe.

It would be advantageous to have a clean, simple and very fast solution to high-level disinfection of ultrasonic transducers.

### BRIEF SUMMARY OF THE INVENTION

The present invention provides a UVC disinfection system which produces a dosage of UVC radiation sufficient to kill Clostridium difficile and other pathogens in a simple fast and unique manner. The system is especially useful for sterilizing and disinfecting medical ultrasound probes or transducers, and can produce more than the necessary dosage of UVC radiation to kill 99% of Clostridium difficile in less than 30 seconds.

The system in one embodiment comprises an enclosure for housing a plurality of UVC lamps which are disposed around a central area in which an ultrasound transducer can be disposed. The lamps are disposed in a vertical orientation, as is the transducer and provide 360° of UVC radiation around the transducer for effective and efficient decontamination. Lamps can also be provided in a horizontal orientation at the bottom of the enclosure for providing UVC radiation in an upward vertical direction, which assures that the tip or outer end of the transducer is always irradiated. The housing has an opening at the top through which the cable of the transducer can extend to hang or suspend the transducer in a vertical orientation in the housing in the center of the surrounding lamps. The opening may have a seal to prevent or minimize leakage of UVC radiation out of the housing. The housing may have a hinged door or panel for access to the lamps for ease of replacement.

The system is controlled by an electronic controller which typically is a microprocessor based controller for control of the ballasts for the UVC lamps and having associated control switches and displays. The ballasts are preferably electronic ballasts which drive the lamps. The current input to the ballasts is monitored by the controller to assure that the UVC lamps are operating properly. If the current is less than the intended reference level, the controller causes turn off of the lamps and display of an error message on a display on the front panel of the housing.

The controller also provides a timer for setting the time period that the UVC lamps are turned on, and monitors safety interlocks to assure that the housing door is closed, and provides various messages and/or indications of system operation.

A UVC radiation sensor can be provided in the housing to monitor radiation intensity from the lamps for comparison by the controller with a reference intensity level to determine that the system is operating properly. If the intensity is below the reference level, the system is shut down by the controller and an appropriate message is displayed.

A UVC sensitive label can also be provided in the housing to monitor total UVC dosage. The color of the label indicates the dosage received by the label and provides an additional means of assuring that the ultrasonic transducer has been properly decontaminated.

### BRIEF DESCRIPTION OF THE SEVERAL VIEWS OF THE DRAWINGS

The invention will be more fully understood from the following detailed description in conjunction with the drawings in which:

FIG. 1 is a pictorial view of one embodiment of the invention;

FIG. 2 is a cutaway pictorial view of the embodiment of FIG. 1;

FIG. 3 is cutaway elevation view of the embodiment of FIGS. 1 and 2 illustrating a transducer in position;

FIG. 4 is a cutaway top view of the embodiments of FIGS. 1-3;

FIG. 5 is cutaway elevation view of another embodiment of the invention;

FIG. 6 is a diagrammatic view of a front or control panel; and

FIG. 7 is a block diagram of a system in accordance with the invention.

### DETAILED DESCRIPTION OF THE INVENTION

This application is a continuation of application Ser. No. 14/521,874 filed Oct. 23, 2014 and is incorporated herein by reference.

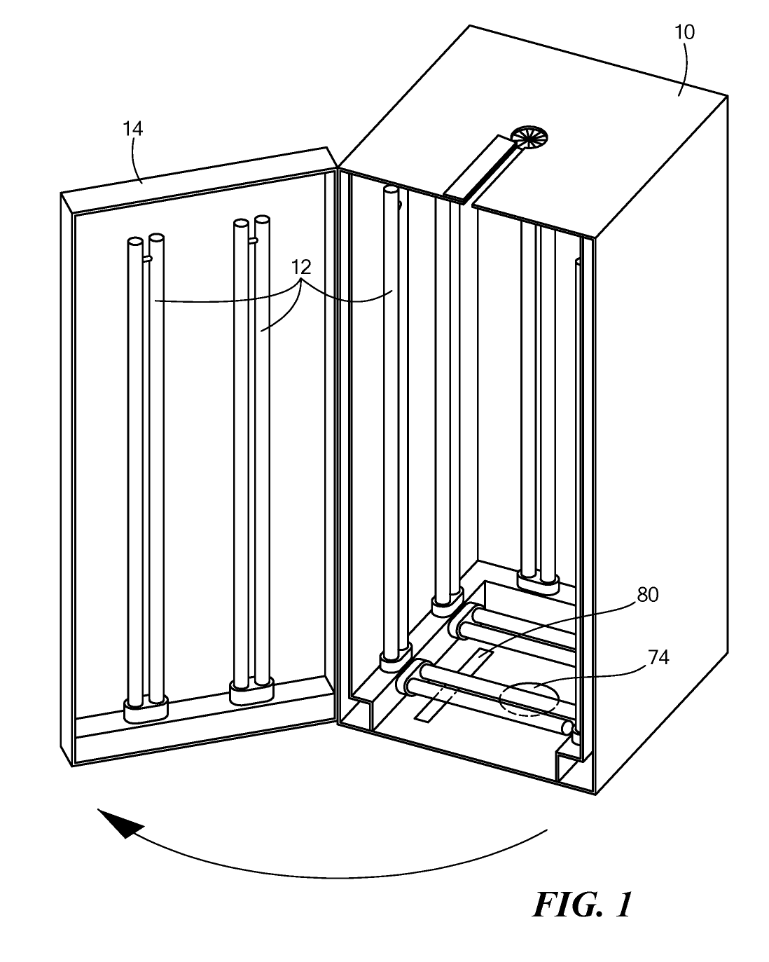

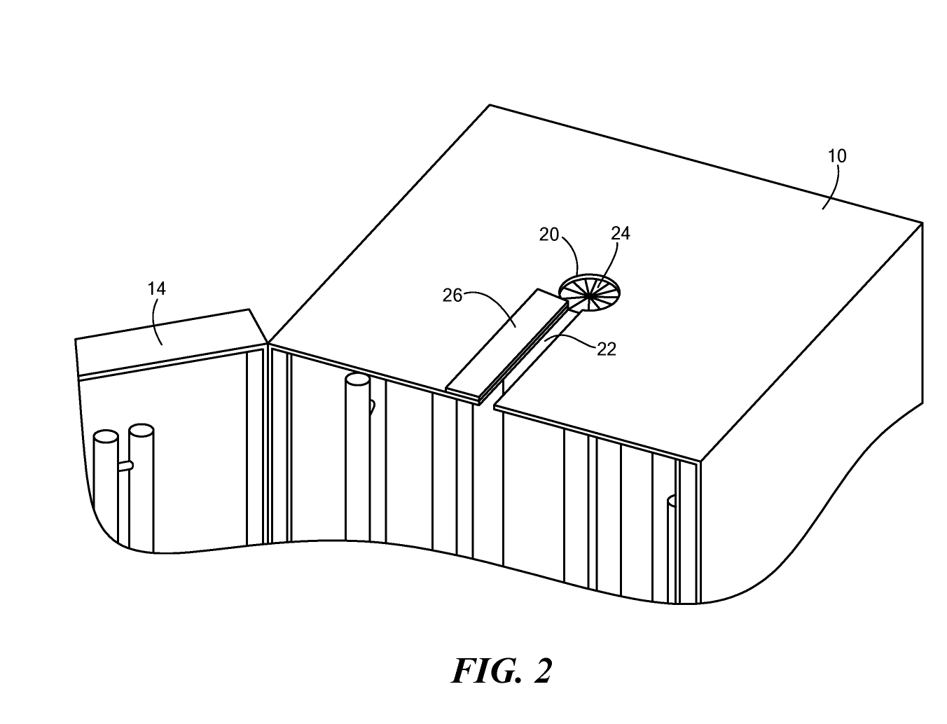

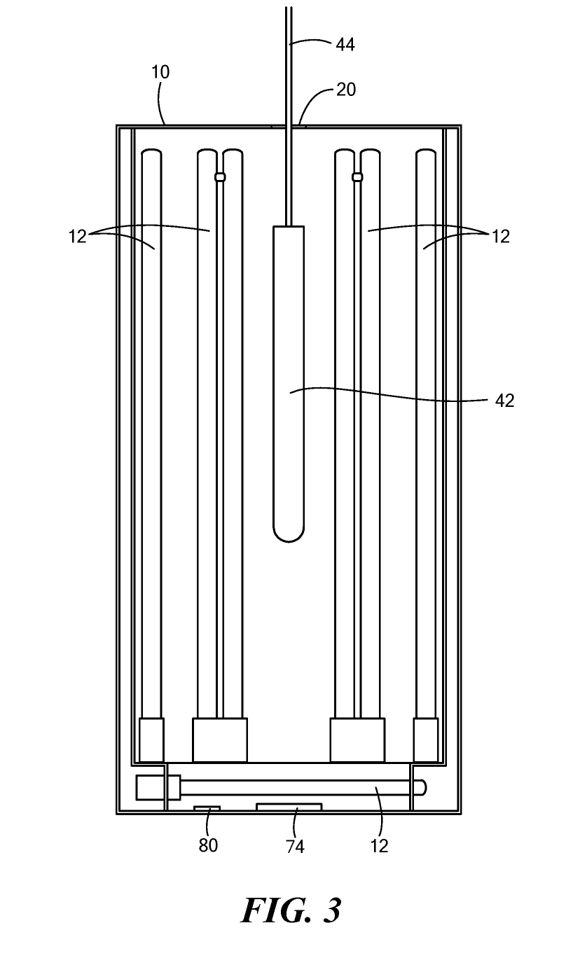

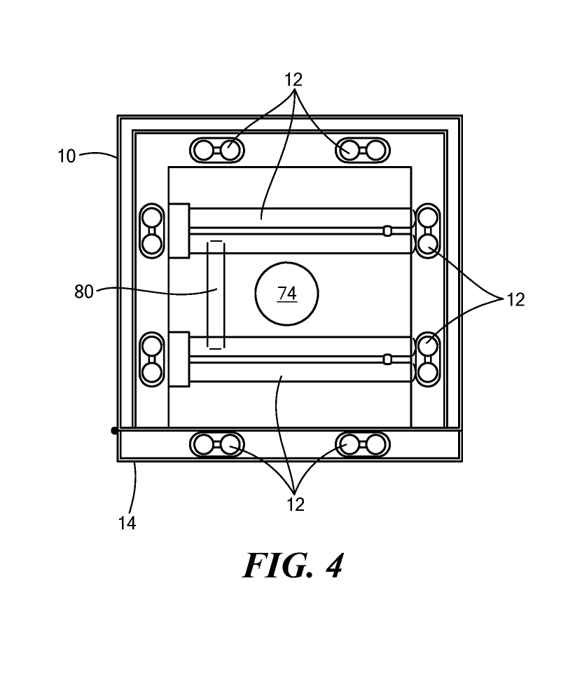

Referring to FIGS. 1-4, one embodiment of a UVC disinfection system in accordance with the invention is shown. A housing or enclosure 10 contains a plurality of UVC lamps 12 disposed about all four sides of the illustrated housing about a central area in which an ultrasonic transducer can be disposed for disinfection. In the illustrated embodiment, the lamps are in the form of U shaped tubes, with two lamps arranged adjacent to each side of the housing. Two of the lamps are in a hinged cover 14 which can swing open as shown in FIG. 2 to provide access to the interior of the housing for insertion and removal of the ultrasonic transducer, and for ease of lamp replacement. One or more lamps are also provided at the housing floor. The lamps at the floor are horizontally disposed and provide UVC radiation upwardly to assure decontamination of the tip of the transducer. The lamps are typically high output, low pressure mercury or amalgam UVC generating lamps, such as Light Sources model LTC 24W/2G11/FEP Coated. The lamps 12 are oriented in a vertical direction to provide UVC radiation which surrounds the central area in which the ultrasonic transducer 42 (FIG. 3) is disposed. The transducer is also placed in a vertical orientation during disinfection, as shown in FIG. 3. The length of the UVC lamps is typically about 12 inches. Preferably the lamps are of U shape so that each can be plugged into electrical sockets in the floor of the housing, as illustrated. The illustrated system produces sufficient UVC radiation to kill 99% of Clostridium difficile in less than 30 seconds.

The housing 10 can be made of any suitable material and in the illustrated embodiment is aluminum which can be oxidized on the interior walls to enhance the reflectance of UVC radiation from the lamps and heighten the efficiency of the system

Preferably each lamp is covered by a protective sleeve to avoid shattering of the lamp glass in the event of breakage. FEP (Teflon) is preferred because it is UVC transmissive with little attenuation and can easily withstand the operating temperature of the UVC lamps.

The top of enclosure 10 has a central opening 20 and a slot 22 which extends from opening 20 to the front edge of the enclosure top. The opening 20 may include a gasket or flexible leaves 24, and the slot may have a cover 26. The cover is open during installation of the transducer in the enclosure and is closed after installation to minimize UVC radiation leakage from the enclosure when the lamps are switched on. The gasket or leaves 24 in the opening 20 also minimize leakage of UVC radiation when the lamps are on.

In the illustrated embodiment, an ultrasound transducer 42 is placed in the housing by sliding the electronic cable 44 of the transducer along the slot 22 of the housing and into the center opening 20. The cover 26 is typically spring loaded and is open during installation of the transducer and closed after the transducer is in place. The slot cover 26 can be linked to the hinged housing door 14 so that the slot cover opens when the housing door is opened. Similarly, the slot cover is closed over the slot when the housing door is closed. The slot is covered to prevent UVC radiation from leaking from the housing. The gasket or flexible leaves 24 of the opening 20 can conform around the electronic cable extending through the opening to prevent or minimize leakage of UVC radiation from the housing. The leaves can be of flexible plastic or rubber which flex to conform around the cable.

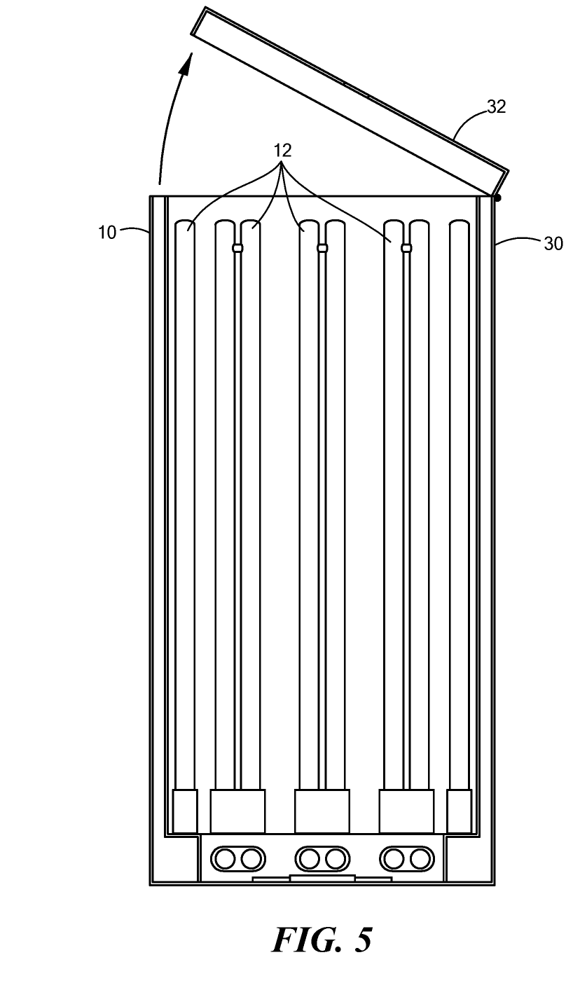

Another embodiment is shown in FIG. 5 and comprises an enclosure 10 having four side walls 30 and a hinged top cover 32. The cover has a slot and a central opening for accommodating the ultrasound transducer, in similar manner to that described in relation to the embodiment of FIGS. 1 and 2. The slot can have a cover and the opening can have a gasket as described above. Three UVC lamps 12 are vertically arranged along each side wall. In addition, three UVC lamps are horizontally disposed at the bottom or floor of the enclosure. The horizontally disposed lamps provide UVC radiation in an upward vertical direction to irradiate the tip of the transducer to further assure its decontamination.

To install the transducer, the cover is opened to permit the cable of the transducer to be slid along the slot to the central location of the top opening to thereby position the transducer in the central area of the enclosure between the vertically oriented UVC lamps.

The UVC lamps are driven by a power source having electronic ballasts which start the lamps and regulate the current in each lamp to assure proper and safe operation. Each lamp may be driven by one electronic ballast or a single ballast may drive up to four lamps depending upon the particular lamps and ballast employed. The electronic ballasts may operate from a standard 110 volt, 60 hertz power source or from a 220 volt, 50 hertz source or from a dual voltage source.

The electronic ballasts are typically controlled by a solid state relay although a mechanical relay may also be used. The system is controlled by a microprocessor based microcontroller typically contained on a control board disposed within the system enclosure. The ballasts and power components can be located, for example, in the bottom of the housing. The control board and associated controls and indicators can be located, for example, behind a control panel which can be located in any convenient position on the enclosure or associated with the enclosure.

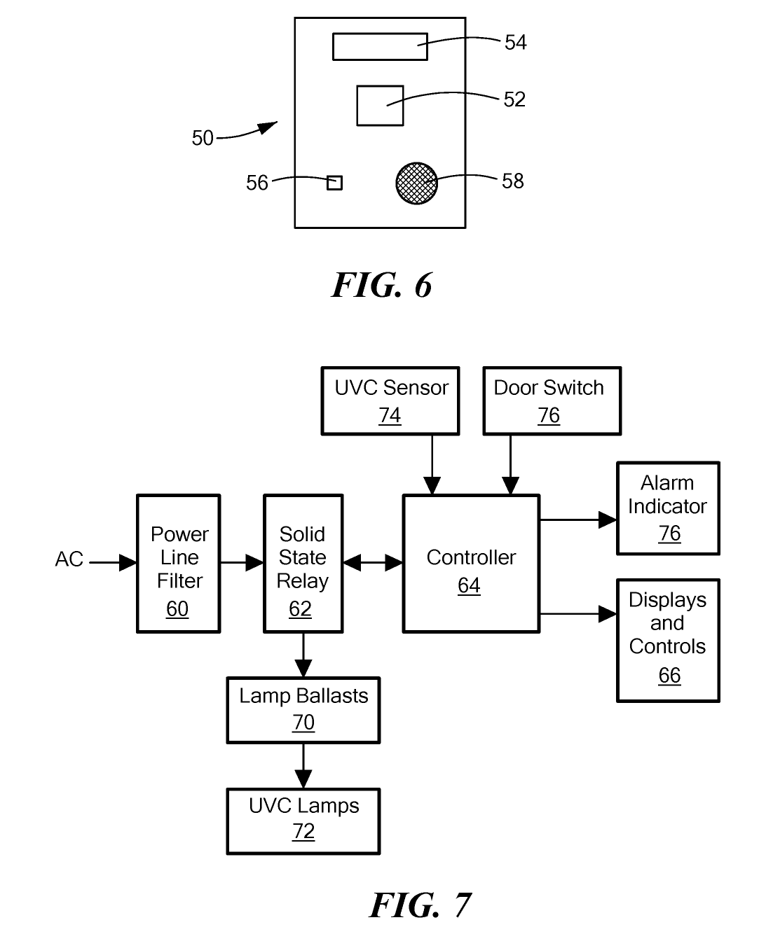

The control panel 50 is illustrated diagrammatically in FIG. 6 and includes a display 52 such as a two digit digital display to indicate countdown of remaining time during a decontamination cycle. A display 54 such as a digital display is provided to show system messages and conditions. A control switch 56 is provided to activate the system. An audio annunciator 58 such as a Sonalert is provided to audibly indicate, such as by a beep, that an operating cycle has ended. The annunciator can also produce distinguishable sounds to denote one or more error conditions.

A block diagram of the system is illustrated in FIG. 7. AC input power is provided via a power line filter 60 and a solid relay 62 to a controller 64 which governs system operation. Over-current protection devices such as a fuse or circuit breaker may be provided. The controller 64 is coupled to displays and controls 66 which include displays or indicators of system conditions and controls for system operation. The controller is also coupled to an alarm indicator 68 which can be an audible and/or visual indicator of alarm conditions. The solid state relay 62 drives the lamp ballasts 70 which drive the UVC lamps 72. A UVC sensor 74 may be provided and coupled to controller 64. The sensor is typically an Indium Gallium Nitride sensor. The UVC sensor receives UVC radiation from the lamps and provides a signal to controller 64 in the event that the sensed UVC radiation falls below a predetermined threshold level, which could occur for example in the event of lamp failure. The UVC sensor 74 is typically connected to an analog-to-digital (AD) converter, so that the measured radiation intensity can be digitally compared with the threshold intensity level to assure that the system is operating properly. The AD converter and comparator can be implemented in the controller or by separate components. The UVC sensor 74 in the embodiment shown in FIG. 1 is disposed in the floor of housing 10, generally in the center between the surrounding UVC lamps 12. A door switch 76 is coupled to controller 64 and in response to an open door, the switch will cause the controller to prevent system operation or shut down operation if the door is opened during an operating cycle. The door can include a locking mechanism which prevents opening of the door during system operation.

The system typically operates for a predetermined period of time as governed by a time period set in the controller. Upon activation of the system, such as by pushing control switch 56, (FIG. 6) the lamps are turned on for the specified time and are turned off when the time period ends.

The controller 64 monitors the current to each of the electronic ballasts 70 to assure that all of the UVC lamps are operating properly. If the current is less than the designated level, the controller will turn off the UVC lamps and display a message on front panel display 54. In order to determine which UVC lamp is not working, the system includes a diagnostic cycle by which the lamps can be turned on when the enclosure door is open. In this manner an operator can see which lamp is not working and have it replaced. A diagnostic cycle can be initiated, for example, by pressing the start button 56 multiple times within a designated time period of time, say five times within five seconds, which will cause all of the lamps to be turned on for visual inspection.

For additional UVC monitoring, a UVC sensitive label 80 may be provided in the housing such as on the floor as shown in FIG. 1 to monitor total UVC dosage. The color of the label indicates the dosage applied and provides additional means of assuring that the ultrasonic transducer was properly decontaminated. Such a label can be for example a Spectra 254 test strip.

Typically the housing is about one foot square and about two feet in height. The gasket 24 in the housing opening 20 can grip the transducer cable with sufficient strength to retain the transducer in hanging position in the housing. Other retention elements or mechanisms can be employed to retain the transducer in proper vertical disposition in the housing between the lamps.

The proper operation of the UVC lamps can be monitored in three ways. The ballast current is monitored by the controller 64 and if the current falls below a predetermined threshold, the lamps are turned off and the system is shut down. Secondly, radiation from the UVC lamps may be monitored by a UVC sensor 74 in the housing, and upon detection of a radiation level below the designated threshold, the system is shut down. In addition a UVC sensitive strip 80 can monitor total dosage of the UVC radiation applied to the transducer to assure that an appropriate dosage has been received for appropriate disinfection.

It will be appreciated that the invention is not to be limited by the particular embodiments shown and that modifications and alternative implementations are contemplated and within the intended scope of the invention. For example, the number and type of UVC lamps can vary and the physical configuration of the system may take different forms. Lamps may not be needed in the floor in some embodiments. Accordingly, the invention is not to be limited by what has been particularly shown and described except as defined by the appended claims.

What is claimed is:

1. An ultraviolet apparatus for disinfecting an ultrasound transducer comprising;
an enclosure having a top, a bottom, four sides and an openable door in one of the sides, for housing a plurality of UVC lamps, the enclosure totally enclosing the ultrasound transducer and preventing any UVC radiation from accidentally leaving the enclosure;
a plurality of UVC lamps providing UVC radiation and having three lamps arranged in a vertical orientation on each of the four sides of the enclosure and three lamps arranged in a horizontal orientation on the bottom of the enclosure to ensure that the entire enclosure is fully flooded with UVC light when the lamps are turned on;
a power source having one or more ballasts to drive said UVC lamps;
an opening in the top of the enclosure which permits an ultrasound transducer to be placed in a vertical orientation in the central area within the surrounding UVC lamps;
wherein the opening in the enclosure includes a slot in the top of the enclosure to permit sliding of an electronic cable of an ultrasound transducer into the center of the top of the enclosure to suspend the transducer in the central area of the enclosure between the UVC lamps; and
an electronic fixed timer to cause the UVC lamps to be turned on for a fixed period of time for a decontamination cycle and having a display to show the time remaining for the UVC lamps to turn off.

2. The system of claim 1 wherein the door is a hinged side door forming a side wall of the enclosure.

3. The system of claim 1 including an electronic controller to turn off the UVC lamps in the event current to the ballasts is less than a reference level, and to display an error message on a display associated with the enclosure.

4. The system of claim 1 including an electronic controller;
a door switch in communication with the electronic controller to indicate the open/closed status of the door;
and wherein the controller is operative to stop or prevent operation of the system if the door is open as signified by the door switch.

5. The system of claim 1 wherein there is a deformable gasket in the opening in the top of the enclosure to prevent the leakage of UVC radiation.

6. The system of claim 1 including a display driven by the controller for indicating the time remaining in a decontamination cycle.

7. The system of claim 1 wherein the UVC lamps are low pressure high output mercury lamps

8. The system of claim 1 wherein the UVC lamps are low pressure high output amalgam lamps.

9. The system of claim 1 wherein the power source includes an over-current protection device.

10. The system of claim 4 wherein the switch can be pressed a predetermined number of times within a fixed period of time to cause the controller to turn on the UVC lamps for a short period of time even with the door open.

11. The system of claim 1 wherein the enclosure is made of aluminum.

12. The system of claim 1 wherein the interior walls of the enclosure are aluminum and oxidized to maximize the reflectance of UVC radiation.

13. The system of claim 1 where the UVC tubes are enclosed in a plastic FEP sleeve to prevent scattering of the UVC tubes in the event of breakage of the tubes.

14. The system of claim 1 including an annunciator to indicate the end of a decontamination cycle.

15. The system of claim 14 wherein the annunciator is a Sonalert.

16. The system of claim 14 wherein the annunciator will emit one signal to indicate the end of a decontamination cycle and a different signal to indicate an error condition.

17. The system of claim 1 including a spring-loaded cover to close the slot to prevent the leakage of UVC radiation from the enclosure.

18. The system of claim 1 wherein there is sufficient UVC intensity to kill 99% of Clostridium difficile (C. Diff) in less than 30 seconds.
